(19) United States
(12) Patent Application Publication
(10) Pub. No.: US 2013/0028976 A1
(43) Pub. Date: Jan. 31, 2013

(54) SPRAY-DRIED CRYSTALLINE ACTIVE INGREDIENT

(71) Applicants: Christopher M. Gregson, Princeton, NJ (US); Ronald H. Skiff, Plainsboro, NJ (US); Ferdinand M. Triolo, Plainsboro, NJ (US)

(72) Inventors: Christopher M. Gregson, Princeton, NJ (US); Ronald H. Skiff, Plainsboro, NJ (US); Ferdinand M. Triolo, Plainsboro, NJ (US)

(73) Assignee: FIRMENICH SA, Geneva 8 (CH)

(21) Appl. No.: 13/637,214

(22) PCT Filed: Mar. 28, 2011
(86) PCT No.: PCT/IB11/51294

§ 371 Date: Sep. 25, 2012

Related U.S. Application Data
(60) Provisional application No. 61/318,529, filed on Mar. 29, 2010.

(51) Int. Cl. A61K 9/14

(52) U.S. Cl. 424/490

(57) ABSTRACT

A process for preparing a spray-dried product for improving the rate of dissolution of the active ingredient. The process includes mixing a crystalline active ingredient, which has a dissolution rate in unstirred water at 25° C. of greater than 15 minutes at a concentration of 14 ppm of the active ingredient, with a starch derivative, a second carrier material, and optionally, xanthan gum, to form a suspension, dispersion or solution of the active ingredient; homogenizing the mixture at a pressure of at least 4000 psig (2.758×10⁷ Pa); and spray-drying the homogenized mixture to provide a spray-dried active ingredient that has an increased dissolution rate in water compared to the unencapsulated active ingredient.

### TECHNICAL FIELD

The present invention relates to a process for preparing a product comprising a crystalline active ingredient in water having excellent dissolution kinetics. The invention also relates to a product produced according to the process.

### BACKGROUND AND PRIOR ART

Many potentially useful active materials are insoluble or at best only sparingly soluble in water. This places significant limits on their usefulness or requires that they are used in combination with solvents which is almost always more expensive than water and which may have physiological incompatibilities or environmental negatives.

In addition, it is desirable for many applications that the active material dissolves rapidly in an aqueous medium. For instance, powdered beverages are diluted with water and consumed immediately, thus requiring all the ingredients to be dissolved rapidly so that the beverage has a balanced flavor. Thus, there is a need to ensure that the active ingredient dissolves sufficiently rapidly.

Various approaches have been taken to improve solubility of poorly soluble materials. A review of these approaches is given in the publication “Formulation approaches for orally administered poorly soluble drugs”, Pinnamaneni S., Das N. G., Das S. K., Pharmazie, 2002, 57, 291 to 300. Such approaches include particle size reduction, modification of the crystal habit, dispersion in carriers, complexation and solubilization by surfactants.

Another review, “Improvement of solubility and dissolution rate of poorly water-soluble salicylic acid by a spray-drying technique” Kawashima Y, Saito M, Takenaka H, J. Pharm. Pharmacol. 1975, 27, 1-5 discloses spray drying a dispersion of salicylic acid in acacia solutions which is said to result in as much as a 50% improvement in the solubility of the product due not only to the concentration of acacia but also the amount of amorphous material in the spray-dried products. The dissolution rate of the spray-dried product is also reported to be almost instantaneous being about 60 times faster than that of the original powder. This is said to be due to a great improvement in the wettability of the spray-dried material.

However, for certain crystalline active ingredients, improved dissolution kinetics may not be sufficiently improved by an increase in wettability.

WO-A-2008/006712 (Unilever) discloses a process for making contra-soluble nano-dispersions of at most sparingly-soluble materials in a soluble carrier material comprising the steps of:
(i) providing a single phase mixture of: (a) a solvent or a mixture of miscible solvents, (b) at least one carrier material soluble in solvent (a), said carrier material being also contra-soluble to payload material (c) and solid at ambient temperature, (c) at least one payload material which is soluble in solvent (a), and,
(ii) drying the mixture to remove solvent (a) and thereby obtain the carrier material (b) in solid form with payload (c) dispersed therein as nanoparticles. Drying is typically performed by spray-drying.

Nevertheless, there remains a strong need to provide a simple process for improving dissolution kinetics.

The present invention seeks to address one or more of the abovementioned problems and/or to provide one or more of the abovementioned benefits.

### SUMMARY OF THE INVENTION

Accordingly, the present invention provides a process for preparing a spray-dried product for improving the rate of dissolution of the active ingredient, the process comprising the steps of:
(i) mixing a crystalline active ingredient, which has a dissolution rate in unstirred water at 25° C. of greater than 15 minutes at a concentration of 14 ppm of the active ingredient, with a starch derivative, a second carrier material, and optionally xanthan gum to form a suspension, dispersion or solution of the active ingredient,
(ii) homogenizing the mixture at a pressure of at least 4000 psig (2.758×10⁷ Pa), and
(iii) spray-drying the homogenized mixture,
   the spray-dried active ingredient having an increased dissolution rate in water compared to the unencapsulated active ingredient.

The invention further provides spray dried particles prepared according to the above mentioned process.

### DETAILED DESCRIPTION OF THE INVENTION

The present invention relates to a process by which the dissolution kinetics of a crystalline active ingredient can be significantly improved.

The invention is based on the combination of certain components, such as compounds or ingredients, together with defined processing conditions.

The first critical component is a starch derivative. By “starch derivative”, it is meant a chemically modified starch, more preferably a hydrophobically modified starch, even more preferably an alkenyl-succinated starch.

The alkenyl-succinated starch preferably has a degree of substitution of from 0.001 to 0.9. The degree of substitution denotes the number of alkenylsuccinic functional groups per glucose units. Thus, a degree of substitution of 0.001 means that there is 1 alkenylsuccinic functional group per 1000 glucose units. The degree of substitution is more preferably from 0.005 to 0.3 and most preferably from 0.01 to 0.1, e.g. from 0.015 to 0.05.

The alkenyl-succinated starch is preferably a C3 to C14 alkenyl-succinated starch, more preferably C4 to C12, most preferably C5 to C10, e.g. C7 to C9.

Most preferably the alkenyl-succinated starch is octenyl-succinated starch. Ideally the octenyl-succinated starch has a degree of substitution no greater than 0.03, more preferably no greater than 0.02.

The dissolution kinetics of the spray dried active ingredient are found to be significantly improved when the starch derivative is present in an amount of from 7% to 25% by weight, more preferably from 7 to 20%, based on the total weight of the mixture prior to spray-drying. At a level of less than 7%, the dissolution rate of the spray-dried active ingredient is hardly improved. Whereas, in an amount greater than 25%, the rate of dissolution is severely adversely affected. Without wishing to be bound by theory, it is believed that the reduction in the rate of dissolution is due to the insolubility of an excess of the starch derivative in water.

Another critical component is the second carrier material. The second carrier material is preferably a carbohydrate. For example, the second carrier material may comprise monosaccharides, disaccharides, trisaccharides, oligosaccharides, polysaccharides or mixtures thereof.

Suitable monosaccharides include: D-Apiose, L-Arabinose, 2-Deoxy-D-ribose, D-Lyxose, 2-O-Methyl-D-xylose, D-Ribose, D-Xylose, which are all pentoses; and Hexoses, such as for instance, L-Fructose, L-galactose, D-Galactose, D-Glucose, D-Mannose, L-Rhamnose, L-Mannose; and mixture of two or more of these.

Mono- and dissacharides may be reduced to the corresponding alcohols, such as for example, xylitol, sorbitol, D-mannitol and/or maltitol. Similarly, oxidation to aldonic, dicaroxyclic acids or uronic acids and reactions with acids, alkalis or amino compounds can give rise to many other compounds like isomaltol, for instance, which may comprise the second carrier material of the present invention.

Suitable oligosaccharides are molecules consisting of from 3 to 10 monosaccharide units, such as maltopentaose, fructo- and/or galacto-oligosaccharides.

Preferably, the second carrier material comprises a maltodextrin, most preferably having a mean dextrose equivalence of 5 to 25, preferably 6 to 20, more preferably 10 to 19.

In a preferred embodiment, the second carrier material is present in an amount of from 40 to 80%, more preferably 60 to 80% by weight, based on the total weight of the mixture prior to spray-drying.

An optional though preferred component is xanthan gum. Xanthan gum is a polysaccharide used as a food additive and rheology modifier. It is produced by fermentation of glucose or sucrose by the Xanthomonas campestris bacterium.

Xanthan gum is preferably added as a processing aid in order to maintain the insoluble crystalline solid suspended in solution prior to spray drying.

The optimum use level for maintaining an adequate suspension was found to be when the xanthan gum is present in an amount of from 0.3% to 0.6% by weight, based on the total weight of the mixture prior to spray-drying. In amounts inferior to 0.3%, it is a noticeable problem that the insoluble solids do not remain suspended in the slurry. Whereas, in amounts superior to 0.6%, the viscosity of the slurry is too high and this can adversely affect the homogenization step.

The active ingredient that is encapsulated is a compound that preferably has a dissolution rate in unstirred water at 25° C. of more than 15 minutes at a concentration of 14 ppm. For the purposes of the present invention, “dissolution rate” is measured according to the method set out in the examples, described below.

The active ingredient is crystalline. By “crystalline” it is meant that the active ingredient forms a structure that exhibits long-range order in three dimensions. Crystallinity can be measured using known techniques in the art such as powder x-ray diffraction (PXRD) crystallography, solid state NMR, or thermal techniques such as differential scanning calorimetry (DSC).

The active ingredient may also be a “sparingly water-soluble organic active agent”. Such an ingredient is typically a compound that has a solubility in water of less than 5% by weight, preferably less than 1% by weight, more preferably less than 0.1% by weight, even more preferably less than 0.01% by weight, in water at 20° C.

Suitable active ingredients include those for use in the foodstuff, pharmaceutical and cosmetic applications. A non-limiting list of examples includes: flavour and fragrance materials of both natural and synthetic origins, compounds and mixtures such as heliotropine, bromelia, (5R5,6R5)-2,6,10,10-tetramethyl-1-oxaspiro-[4,5]dec-6-yl acetate, acetanisole, methylsalicylique aldehyde, para-ethyl phenol, phenol, phenylethyl salicylate, menthol, cyclohexanecarboxamide, veratraldehyde, xylenol, dodecanoic acid, thymol, heliotropyl acetate, methyl anisate, methylnaphtylketone, myristic acid, palmitic acid, dimethylphenol, dimethyl acrylic acid, coumarine, methyl cyclopentenolone, 7-methylcoumarin, phenylacetate, acetylpyrazine, phenylacetic acid, isoeugenyl acetate, raspberry ketone, naringin, propenyl guaethol, tetramethylpyrazine, acetylmethyl carbinol, 3-hydroxy-2-ethyl-4-pyranone, malic acid, resorcinol, benzoic acid, cinnamic acid, benjoin sumatra, benjoin siam, citric acid, tartric acid, camphor, quinine chlorohydrate, ascorbic acid, borneol, glutamic acid, 5-methyl quinoxaline and malt extract; liposoluble vitamins, such as vitamin A and derivatives, vitamin D₂, vitamin D₃, natural or synthetic α-, β-, γ- or δ-tocopherol, preferably natural or synthetic α-tocopherol, and tocotrienol and tocopheryl C₁-C₂O-carboxylates; water-insoluble or sparingly water-soluble organic UV screening substances, such as, e.g., compounds from the group of the triazines, anilides, benzophenones, triazoles, cinnamides and sulfonated benzimidazoles; polyunsaturated fatty acids, such as, e.g., arachidonic acid, eicosapentaenoic acid or docosahexaenoic acid; food colorants, such as curcumine, carmine or chlorophyll; carotenoids, such as, e.g., β-carotene and lycopene; and xanthophylls, such as, e.g. lutein, astaxanthin, zeaxanthin, capsanthin, capsorubin, cryptoxanthin, citranaxanthin, canthaxanthin, bixin, β-apo-4-carotenal, β-apo-8-carotenal and β-apo-8-carotenic acid ethyl ester. The technical field of application is not critical though.

The active ingredient is preferably present in an amount of from 15 to 25% by weight, based on the total weight of the spray dried particle.

A suitable process for producing the spray-dried particles of the invention comprises a first step of forming a dispersion, suspension or solution of the starch and second carrier material. This may be performed by simple mixing of the components with water, preferably hot water at a temperature of 60° C. to 80° C. Separately, a dispersion or solution is prepared of the active ingredient in a suitable solvent, such as propylene glycol. The two solutions are then mixed together.

The resulting mixture is homogenized according to defined processing parameters in order to provide a product having excellent dissolution kinetics.

Critically, the spray dried particles are prepared by a process in which a homogenization step occurs at a minimum pressure of 4000 psig, more preferably 5000 psig or more, most preferably 5500 psig or more prior to the spray drying step.

The homogenization step is preferably carried out in two or more complete passes in a standard homogenizer. A two-stage homogenizer is suitable for this purpose.

After homogenization, the mixture is subjected to a spray-drying step. The spray drying is preferably performed using nozzle atomization only. The spray pressure is preferably from about 1500 to 2500 psig. The inlet temperature is preferably from 180° C. to 200° C. The outlet temperature is preferably from 70° C. to 100° C.

The spray dried particles can then be collected by any standard process. Optionally and preferably they are passed through a sieve, such as a 20 Mesh screen to obtain a more homogeneous particle size.

The spray dried particles provide a significantly improved dissolution rate in a 0.05% aqueous citric acid solution of the active ingredient compared to the dissolution rate of the unencapsulated active ingredient. Further, most of the spray dried particles have an improved dissolution rate in water of the active ingredient compared to the dissolution rate of the unencapsulated active ingredient.

For instance, the dissolution rate in a 0.05% aqueous citric acid solution of the spray dried particles at 25° C. is preferably less than 15 minutes at a concentration of 14 ppm of the active ingredient, more preferably less than 10 minutes, even more preferably less than 4 minutes, most preferably less than 2 minutes.

### EXAMPLES

The invention will now be described with reference to the following examples. It is to be understood that the examples are illustrative of the invention and that the scope of the invention is not limited thereto.

Samples according to the invention are denoted by a number and comparative examples by a letter.

In the examples, and more generally for the purposes of the present invention, calculation of the dissolution kinetics of the spray-dried active ingredient are performed as follows.

Detection was made using a fiber optic spectrometer, comprising a D₂Lite deuterium tungsten light source, TR 600-10 transmission probes (10 mm pathlength tip) and a 52000 spectrometer (World Precision Instruments/Ocean Optics). Data was recorded using the Ocean Optics software OOIBase32. Acquisition rate was set at one measurement per second.

The spectrum of the fully dissolved active was initially determined in the appropriate solvent (water or citric acid aq.) to find an appropriate wavelength for measurements. An amount of the spray dried powder was used for each experiment resulting in an equivalent nominal concentration of 14 ppm of the active ingredient. The λmax at 324 nm was chosen as the signal wavelength. A second wavelength (400 nm) where no absorption by the active ingredient was detected was used to correct the signal for changes in baseline caused by particles, turbidity, etc.

The spray dried powders were weighed on a weighing paper and rapidly tipped into the dissolution vessel (Distek 2100B, 1 liter flask maintained at 25° C. using a water-bath) containing deionized water as the solvent for one set of experiments, and 500 ppm citric acid in deionized water for a second set of experiments. The contents were stirred using paddles at 200 rpm. Experiments were run until there was no longer a noticeable increase in absorbance on a plot of absorbance versus log time. The results were normalized by dividing the absorbance at each data point with the maximum absorbance. The kinetics of dissolution were quantified by finding the time at which the normalized absorbance reached 0.95. These values are equivalent to the dissolution of 95% of the active ingredient.

The same method was used for calculating the dissolution rate of the active ingredient alone.

### Example 1

### Preparation of Spray-Dried Particles

The compositions given in table 1 were prepared as follows. The xanthan gum, maltodextrin and starch were added to the water that had been preheated to 70° C., and mixed until fully dissolved. Separately, the active ingredient was added to the propylene glycol solution and mixed for 30 minutes. The two mixtures were then combined and mixing was continued for a further 30 minutes.

The mixture was then transferred to a high pressure homogenizer and homogenization was carried out in two passes at the pressures given in the following table. The homogenized product was then spray dried under standard conditions at an inlet temperature of 171° C. and an outlet temperature of 82° C. The resulting spray-dried particles were collected as a yellow powder. All amounts are parts by weight.

TABLE 1

| | | | | Active | Starch | | Homogenization | |
|---|---|---|---|---|---|---|---|---|
| | PG | Xanthan | Maltodextrin | Ingredient | Derivative | | Pressure (psig) | |
| Sample | (1) | Gum (2) | 18DE (3) | (4) | (5) | Water | 1^{st} pass | 2^{nd} pass |
| 1 | 4.48 | 0.4 | 71.46 | 15.7 | 7.96 | 158 | 5800 | 5800 |
| 2 | 4.48 | 0.4 | 71.46 | 15.7 | 7.96 | 158 | 5800 | 5800 |
| 3 | 4.48 | 0.4 | 75.71 | 11 | 8.41 | 158 | 5800 | 5800 |
| 4 | 4.48 | 0.4 | 75.71 | 11 | 8.41 | 158 | 5800 | 5800 |
| 5 | 4.48 | 0.4 | 57.42 | 15.7 | 22 | 158 | 5800 | 5800 |
| 6 | 4.48 | 0.4 | 53.12 | 20 | 22 | 158 | 5800 | 5800 |
| 7 | 4.48 | 0.4 | 67.61 | 20 | 7.51 | 158 | 5800 | 5800 |
| 8 | 4.48 | 0.4 | 53.12 | 20 | 22 | 158 | 4000 | 4000 |
| 9 | 4.48 | 0.4 | 45.12 | 20 | 30 | 158 | 4000 | 4000 |

(1) Propylene glycol
(2) ex Firmenich, ref. 946867
(3) Glucidex 19D, ex Roquette Frères
(4) Poorly soluble crystalline taste-modifying active ingredient, ex Firmenich ref. 983950
(5) Capsul, ex National Starch

The samples were then dissolved in water or an aqueous citric acid solution, as described above to provide a solution containing 14 ppm of the active ingredient.

TABLE 2

| | Gram added per liter solution (to provide 14 ppm |
|---|---|
| Sample | active ingredient) |
| 1 | 0.089 |
| 2 | 0.089 |
| 3 | 0.127 |
| 4 | 0.127 |
| 5 | 0.089 |
| 6 | 0.07 |
| 7 | 0.07 |
| 8 | 0.07 |
| 9 | 0.07 |

The rate of dissolution of the various samples was then evaluated according to the methodology described above. The results for 95% dissolution are given in the following tables:

TABLE 3

| | In water (sec) | | | | |
|---|---|---|---|---|---|
| Sample | trial 1 | trial 2 | trial 3 | trial 4 | average |
| 1 | 925 | 853 | — | — | 889 |
| 2 | 749 | 665 | — | — | 707 |
| 3 | 573 | 599 | — | — | 586 |
| 4 | 479 | 556 | — | — | 518 |
| 5 | 1162 | 1300 | — | — | 1231 |
| 6 | 721 | 818 | 802 | 622 | 741 |
| 7 | 762 | 1310 | — | — | 1036 |
| 8 | 1334 | 1184 | 1148 | 1215 | 1220 |
| 9 | 2071 | 2188 | — | — | 2130 |

TABLE 4

| | In 0.05% citric | | | | |
|---|---|---|---|---|---|
| | acid aqueous solution (sec) | | | | |
| Sample | trial 1 | trial 2 | trial 3 | trial 4 | average |
| 1 | 230 | — | — | — | 230 |
| 2 | 228 | — | — | — | 228 |
| 3 | 78 | — | — | — | 78 |
| 4 | 113 | — | — | — | 113 |
| 5 | 154 | — | — | — | 154 |
| 6 | 113 | 165 | 130 | — | 136 |
| 7 | 111 | — | — | — | 111 |
| 8 | 193 | 144 | 228 | 311 | 219 |
| 9 | 220 | 323 | — | — | 272 |

The active ingredient alone had a dissolution rate of 1106 seconds in water at 25° C. and of 437 seconds in 0.05% aqueous citric acid solution. Thus, the spray dried systems comprising the active ingredient show a remarkable improvement in dissolution kinetics in all cases when the spray dried active ingredient was dissolved in a dilute aqueous citric acid solution and in most cases where the spray dried active ingredient was dissolved in water.

1-13. (canceled)

14. A process for improving the rate of dissolution in water of an active ingredient, which comprises:
mixing a crystalline active ingredient, which has a dissolution rate in unstirred water at 25° C. of greater than 15 minutes at a concentration of 14 ppm of the active ingredient, with a starch derivative, a second carrier material, and optionally xanthan gum to form a suspension, dispersion or solution of the active ingredient;
homogenizing the mixture at a pressure of at least 4000 psig (2.758×10⁷ Pa); and
spray-drying the homogenized mixture to provide the spray-dried active ingredient with an increased dissolution rate in water compared to the unencapsulated active ingredient.

15. The process according to claim 14, wherein the starch derivative is present in an amount of from 7 to 25% by weight, based on the total weight of the spray dried particle.

16. The process according to claim 14, wherein the amount of xanthan gum is from 0.3 to 0.6% by weight, based on the total weight of the mixture prior to spray-drying.

17. The process according to claim 14, wherein the active ingredient is present in an amount of from 3% to 25% by weight, based on the total weight of the mixture prior to spray-drying.

18. The process according to claim 14, wherein the homogenization step is performed in two or more passes through a homogenizer.

19. The process according to claim 14, wherein the starch derivative comprises a C3 to C14 alkenyl-succinated starch.

20. The process according to claim 19, wherein the alkenyl-succinated starch is an octenyl-succinated starch.

21. The process according to claim 20, wherein the octenyl-succinated starch has a degree of substitution no greater than 0.03.

22. The process according to claim 14, wherein the second carrier material comprises a maltodextrin having a mean dextrose equivalence of 5 to 25.

23. The process according to claim 14, wherein the homogenizing is conducted in two complete passes.

24. The process according to claim 14, wherein the starch derivative is present in an amount of from 7 to 25% by weight, based on the total weight of the mixture prior to spray-drying.

25. The process according to claim 14, wherein the second carrier material is present in an amount of from 60 to 80% by weight, based on the total weight of the mixture prior to spray-drying.

26. The process according to claim 14, wherein the dissolution rate of the active ingredient at a concentration of 14 ppm in unstirred water at 25° C. is improved by reducing dissolution time from 1106 seconds to no more than 889 seconds.

27. The process according to claim 14, wherein the dissolution rate of the active ingredient at a concentration of 14 ppm in unstirred water at 25° C. is improved by reducing dissolution time from 1106 seconds to no more than 707 seconds.

28. The process according to claim 14, wherein the dissolution rate of the active ingredient at a concentration of 14 ppm in an unstirred aqueous solution of water containing 0.05% citric acid at 25° C. is improved by reducing dissolution time from 437 seconds to no more than 219 seconds.

29. The process according to claim 14, wherein the dissolution rate of the active ingredient at a concentration of 14 ppm in an unstirred aqueous solution of water containing 0.05% citric acid at 25° C. is improved by reducing dissolution time from 437 seconds to no more than 154 seconds.

30. A spray dried particle prepared according to the process of claim 14.
